(19) United States
(12) Patent Application Publication
(10) Pub. No.: US 2008/0135641 A1
(43) Pub. Date: Jun. 12, 2008

(54) Support member and volatilizing apparatus

(76) Inventors: Tomonori Iwasaki, Sanda-shi (JP); Osamu Matsumoto, Toyonaka-shi (JP)

Correspondence Address: WENDEROTH, LIND & PONACK, L.L.P., 2033 K STREET N. W., SUITE 800, WASHINGTON, DC, 20006-1021, US

(21) Appl. No.: 12/010,835

(22) Filed: Jan. 30, 2008

Related U.S. Application Data
(62) Division of application No. 11/033,846, filed on Jan. 13, 2005.

(30) Foreign Application Priority Data
Jan. 30, 2004 (JP) P2004-023107

(51) Int. Cl. A61L 9/04

(52) U.S. Cl. 239/53

(57) ABSTRACT

A support member is comprised of a carrier which holds a volatile component. The carrier is comprised of a three-dimensional knitted fabric. The three-dimensional knitted fabric is comprised of a first knitted fabric layer, a second knitted fabric layer, and connecting yarns between these layers. The connecting yarns connect the first knitted fabric layer with the second knitted fabric layer. At least one knitted fabric layer has a mesh-like structure. A plurality of connecting yarns extend from each stitch of the first knitted fabric layer to each stitch of the second knitted fabric layer and connect each other's stitch. The volatile component is impregnated in the carrier or adhered to the carrier, so that the volatile component is held by the carrier.

### BACKGROUND OF THE INVENTION

The present invention relates to a support member for a volatile component, and a volatilizing apparatus including the support member.

Now, an insect-pest-control apparatus of a non-heating type is remarkable compared to a conventional apparatus of a heating-type since the apparatus of the non-heating type does not need heating and also is suitable for use indoor and outdoor. The apparatus of the non-heating type includes a volatile insect-pest-control component, such as a volatile insecticidal component, a volatile insect-pest-repellent component and the like, as a volatile component.

A fan-type apparatus is proposed as the apparatus of the non-heating type. For example, the fan-type apparatus is disclosed in JP-A No. 7-236399. In the fan-type apparatus, a fan blows air to a support member, so that a volatile component is released from the support member into an atmosphere. The support member is comprised of a carrier such as a corrugated paper and the like. The volatile component is impregnated in the carrier.

For example, a support member which is used for an insect-pest-control apparatus of a non-heating type is disclosed in JP-A No. 2001-200239. A support member of this apparatus is constructed with overlapped nets. Each net is made of twisted yarns which hold the volatile component.

### SUMMARY OF THE INVENTION

However, the insect-pest-control apparatus including the above-mentioned support member does not exert sufficiently an insect-pest-control effect immediately after beginning of use. Therefore, the above-mentioned support member can not exert sufficiently an efficacy of the volatile component.

An objective of the present invention is to provide a support member which can release efficiently a volatile component to exert sufficiently efficacy of the volatile component from a beginning stage, and is to provide a volatilizing apparatus which includes the support member.

As a result of repeating examination ardently, the inventors of the present application have determined that the above-mentioned objective could be achieved by using a three-dimensional knitted fabric of a specific structure as a carrier for holding the volatile component.

A support member of the present invention is as follows. The support member is comprised of a carrier which holds a volatile component. The carrier is comprised of a three-dimensional knitted fabric. The three-dimensional knitted fabric is comprised of a first knitted fabric layer, a second knitted fabric layer, and connecting yarns between these layers. The connecting yarns connect the first knitted fabric layer with the second knitted fabric layer. At least one knitted fabric layer has a mesh-like structure. A plurality of connecting yarns extend from each stitch of the first knitted fabric layer to each stitch of the second knitted fabric layer and connect each other's stitch. The volatile component is impregnated in the carrier or adhered to the carrier, so that the volatile component is held by the carrier.

According to the support member of the present invention, the volatile component can be released efficiently to exert sufficiently efficacy of the volatile component from the beginning stage. Therefore, the support member is very useful for an insect-pest-control apparatus of a non-heating type, particularly a fan-type apparatus.

A volatilizing apparatus of the present invention is as follows. A volatilizing apparatus is comprised of the support member of the present invention and a fan for blowing air to the support member.

According to the volatilizing apparatus, efficacy of the volatile component can be exerted remarkably from the beginning stage.

### BRIEF DESCRIPTION OF THE DRAWINGS

FIG. 1 is an enlarged view showing a first example of a three-dimensional knitted fabric of the present invention.

FIG. 2 is an enlarged view showing a second example of the three-dimensional knitted fabric of the present invention.

FIG. 3 is an enlarged view showing a third example of the three-dimensional knitted fabric of the present invention.

FIG. 4 is a perspective view showing a test apparatus.

### DETAILED DESCRIPTION OF THE PREFERRED EMBODIMENTS

The term, “a mesh-like knitted fabric”, means a knitted fabric having a plurality of openings, such as a mesh knitted fabric, a marquisette knitted fabric and the like.

A support member of the present invention is comprised of a carrier which holds a volatile component. The carrier is comprised of a three-dimensional knitted fabric. The three-dimensional knitted fabric is comprised of a first knitted fabric layer, a second knitted fabric layer, and connecting yarns between these layers. The connecting yarns connect the first knitted fabric layer with the second knitted fabric layer. At least one knitted fabric layer has a mesh-like structure. A plurality of connecting yarns extend from each stitch of the first knitted fabric layer to each stitch of the second knitted fabric layer and connect each other's stitch. The volatile component is impregnated in the carrier or adhered to the carrier, so that the volatile component is held by the carrier.

In the present invention, at least one knitted fabric layer requires a mesh-like structure. Two knitted fabric layers may be the same or different mesh-like knitted fabric layers.

At least one knitted fabric layer is preferably knitted with a bundle of at least two yarns.

It is required that a plurality of connecting yarns extend from each stitch of the mesh-like knitted fabric layer to each stitch of the other knitted fabric layer and connect each other's stitch. It is preferred that these connected stitches do not face each other. The connecting yarns extend so as to play a role in keeping a distance between two knitted fabric layers. Therefore, as long as the connecting yarns can keep the distance between two knitted fabric layers, the connecting yarns may be disposed at a slant or per saltire.

It is preferred that, in order to keep the distance between two knitted fabric layers and hold sufficiently the volatile component, a plurality of connecting yarns extend from each stitch of all stitches of the mesh-like knitted fabric layer to each stitch of the other knitted fabric layer and connect each other's stitch. These connected stitches do not face each other. It is preferred that a yarn made of a monofilament is used as a connecting yarn from a viewpoint of enhancing strength of the carrier. The connecting yarns may be formed into a loop-like stitch in two knitted fabric layers. The connecting yarns may be hooked to a stitch of two knitted fabric layers with tuck structure.

In case that a number of connecting yarns is one per one stitch of the mesh-like knitted fabric layer, there are risks as follows. That is, a distance between two knitted fabric layers cannot be kept, and efficacy of the support member cannot be exerted sufficiently since an amount of the volatile component to be held is insufficient.

Concrete examples of three-dimensional knitted fabrics which are used as carriers are shown in FIGS. 1, 2, and 3 respectively. These figures are model figures.

In carrier 1 shown in FIG. 1, two connecting yarns 4 extend from each stitch of all stitches of mesh-like knitted fabric layer 2 to each stitch of mesh-like knitted fabric layer 3. Each stitch of layer 2 is connected with each stitch of the layer 3 by two connecting yarns 4. These connected stitches do not face each other.

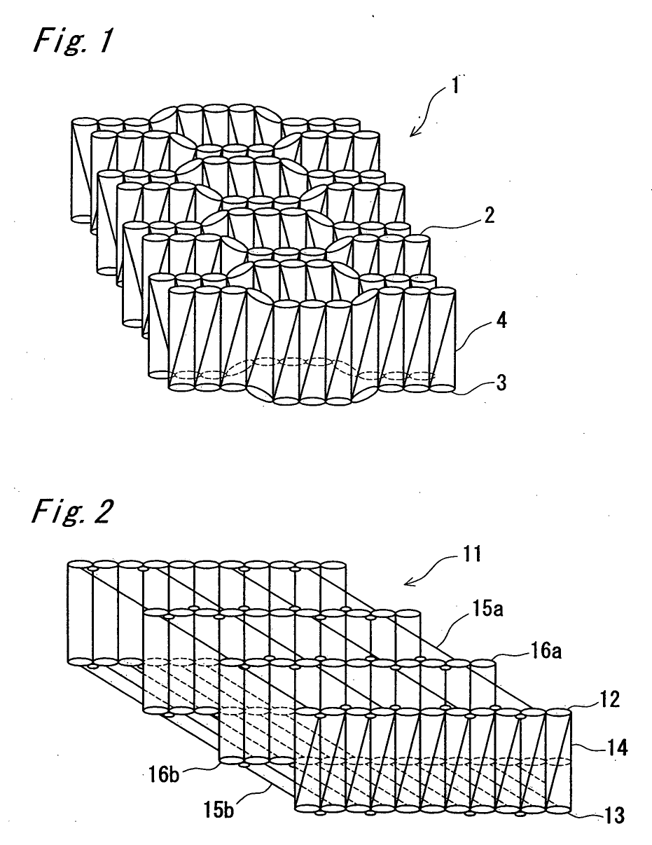

In carrier 11 shown in FIG. 2, two connecting yarns 14 extend from each stitch of all stitches of mesh-like knitted fabric layer 12 to each stitch of knitted fabric layer 13. Each stitch of layer 12 is connected with each stitch of layer 13 by two connecting yarns 14. These connected stitches do not face each other. The mesh-like knitted fabric layer 12 is constructed with chain knitting yarns 16a and insert yarns 15a. The knitted fabric layer 13 is constructed with chain knitting yarns 16b and insert yarns 15b.

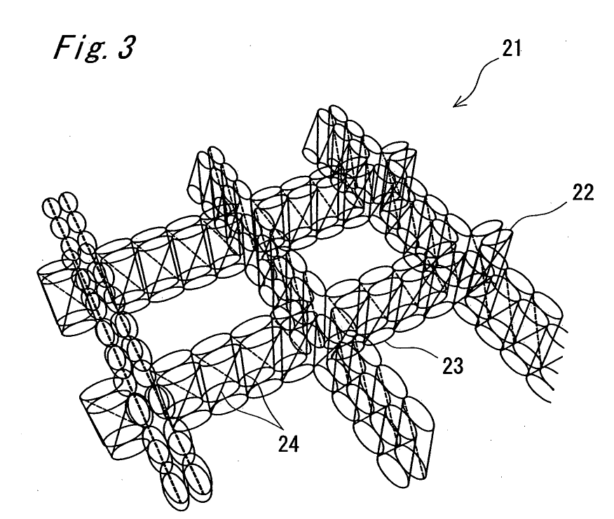

In carrier 21 shown in FIG. 3, two connecting yarns 24 extend from each stitch of all stitches of mesh-like knitted fabric layer 22 to each stitch of mesh-like knitted fabric layer 23. Each stitch of layer 22 is connected with each stitch of layer 23 by two connecting yarns 24. These connected stitches do not face each other.

It is preferred that, in order that the carrier has appropriate elasticity or appropriate repulsion, a width of the connecting yarn is 15˜2000 denier, preferably 50˜300 denier, in single yarn denier.

This carrier is usually a knitted fabric having a double needle bed. The knitted fabric is a longitude knitted fabric or a latitude knitted fabric. For example, this carrier can be prepared by a double-raschel machine or a double-circular-knitting machine and the like. This three-dimensional knitted fabric of this carrier can be made of the following fibers. For example, synthetic fibers such as polyamide, polyacrylonitrile and the like; semi-synthetic fibers such as acetate, triacetate and the like; regenerated fibers such as rayon, cupra and the like; natural fibers such as wool, cotton and the like. Polyamide is more preferable among these fibers since it is superior to chemical resistance and rigidity of structural formation.

A thickness of this carrier, that is, a distance between two knitted fabric layers, is preferably 2 mm˜mm, A unit weight of this carrier is usually 50 g/m²˜2.5 kg/m², preferably 200 g/m²˜1000 g/m².

For example, FUSION (Trademark; Distributor: ASAHI KASEI FIBERS CORPORATION) can be used as this carrier. Model number: AKE69440 among FUSION can be used preferably as this carrier. Model number: AKE69440 has a three-dimensional knitted structure shown in FIG. 3. In the present invention, these three-dimensional knitted fabrics which are commercially available can be used as this carrier without change.

This carrier is provided for use after cutting the fabric into a desired size. Alternatively, this carrier is provided for use after cutting the fabric and then sewing or thermoforming it into a predetermined form.

This support member can be obtained by making this carrier hold a volatile component. Following methods can be used for making this carrier hold the volatile component. One method has a step of impregnating the carrier with the volatile component or a volatile solution and then drying the carrier if necessary. The volatile solution is comprised of a suitable solvent in which the volatile component is dissolved. Another method has a step of applying the volatile component or the volatile solution to the carrier and then drying the carrier if necessary.

In this carrier, the volatile component may be held by either or both of two knitted fabric layers.

Compounds which can volatilize at an ordinary temperature (for example, a vapor pressure at 25° C. is at least 1×10⁻⁶ mm Hg) and have physiological activity (for example, perfume activity, insect-pest-control activity) can be used as the volatile component to be held by this carrier. When this carrier is used for an insect-pest-control apparatus of a non-heating type, especially an apparatus of a fan-type, an effect of this carrier is remarkable. It is preferred to use a volatile insect-pest-control component as the volatile component. It is preferred to use an insect-pest-control active compound which can volatilize at an ordinary temperature (for example, a vapor pressure at 25° C. is at least 1×10⁻⁶ mmHg), as the volatile insect-pest-control component which is used in the present invention.

Following compounds can be used as the above-mentioned insect-pest-control active compound.
5-propargyl-2-furfuryl2,2,3,3-tetramethylcyclopropanecarboxylate,
1-ethynyl-2-methyl-2-pentenyl3-(2-methyl-1-propenyl)-2,2-dimethylcyclopropanecarboxylate,
1-ethynyl-2-methyl-2-pentenyl3-(2-chloro-2-fluorovinyl)-2,2-dimethylcyclopropane-1-carboxylate,
2,3,5,6-tetrafluoro-4-methylbenzyl3-(2-methyl-1-propenyl)-2,2-dimethylcyclopropanecarboxylate,
2,3,5,6-tetrafluoro-4-methylbenzyl3-(2-chloro-2-fluorovinyl)-2,2-dimethylcyclopropanecarboxylate,
2,3,5,6-tetrafluorobenzyl3-(2,2-dichlorovinyl)-2,2-dimethylcyclopropanecarboxylate,
2,3,5,6-tetrafluoro-4-methylbenzyl3-(1-propenyl)-2,2-dimethylcyclopropanecarboxylate,
2,3,5,6-tetrafluoro-4-methylbenzyl3-(2,2-dichlorovinyl)-2,2-dimethylcyclopropanecarboxylate,
2,3,5,6-tetrafluoro-4-methoxymethylbenzyl3-(2-methyl-1-propenyl)-2,2-dimethylcyclopropanecarboxylate,
2,3,5,6-tetrafluoro-4-methoxymethyl benzyl 3-(1-propenyl)-2,2-dimethylcyclopropanecarboxylate,
2-methyl-3-allyl-4-oxo-2-cyclopenten-1-yl2,2,3,3-tetramethylcyclopropanecarboxylate,
natural pyrethrin.

In the present invention, only one kind of the above-mentioned compounds may be used, or two or more kinds of the above-mentioned compounds may be used by mixing these compounds.

From viewpoints of insect-pest-control activity and volatile property, at least one kind of compound is preferably selected from the group consisting of the following compounds.
2,3,5,6-tetrafluoro-4-methoxymethylbenzyl3-(1-propenyl)-2,2-dimethylcyclopropanecarboxylate,
2,3,5,6-tetrafluoro-4-methylbenzyl3-(1-propenyl)-2,2-dimethylcyclopropanecarboxylate,
2,3,5,6-tetrafluorobenzyl3-(2,2-dichlorovinyl)-2,2-dimethylcyclopropanecarboxylate.

Moreover, at least one kind of compound is preferably selected from the group consisting of the following compounds.
2,3,5,6-tetrafluoro-4-methoxymethylbenzyl (1R)-trans-3-(1-propenyl(Z/E=8/1))-2,2-dimethylcyclopropanecarboxylate,
2,3,5,6-tetrafluoro-4-methylbenzyl (1R)-trans-3-(1-propenyl(Z/E=8/1))-2,2-dimethylcyclopropanecarboxylate.

An a mount of the volatile component held in t his support member can be changed according to application, status of use, or duration of use and the like. Generally, a range of the above-mentioned amount is 0.001 g˜10 g, preferably 0.01 g˜5 g, more preferably 0.05 g˜1 g, per 0.5 g of the carrier.

The following compounds or components other than the insect-pest-control active compound can be used as the volatile component. For example, a perfume-antibacterial-insect-pest-repellent component which is contained in vegetable essential oil and the like, or a synthetic insect-pest-repellent active compound such as Deet (Trade Name of diethyltoluamide) and the like can be used. In the present invention, a plurality of volatile components can be used at the same time. In that case, the volatile insect-pest-control active compound can be held by one knitted fabric layer and the volatile insect-pest-repellent active compound can be held by the other knitted fabric layer.

If an antioxidant, such as BHT, or an ultraviolet absorbent, is added to the volatile component, stability of the support member to light, heat, or oxidation can be increased.

This support member can be used in the fan-type volatilizing apparatus and can exert an effect of desired insect-pest-control. In that case, this support member is reinforced with a suitable reinforcing material if necessary and is equipped in a place where airflow will occur in an upwind or downwind side of the fan. And then, the fan is rotated. In the above-mentioned fan-type volatilizing apparatus, speed of airflow passing through the support member is usually 0.1 m/s˜10 m/s.

The present invention will be described below in further detail by way of examples. The present invention should not be limited to these examples.

### Example 1

First, a three-dimensional knitted fabric (Trade Name: FUSION; Model Number: AKE69440; Distributor: ASAHI KASEI FIBERS CORPORATION; Thickness: 4.3 mm; Unit Weight: 321 g/m²; made from polyamide) which has knitted structure shown in FIG. 3 was cut into a circular form having a diameter of 5 cm. Next, an acetone solution of 120 mg of 2,3,5,6-tetrafluoro-4-methoxymethylbenzyl(1R)-trans-3-(1-propenyl(Z/E=8/1))-2,2-dimethylcyclopropanecarboxylate was applied uniformly to the three-dimensional knitted fabric. And then, acetone was air-dried to prepare support member 101 of the present invention.

### Example 2

First, a three-dimensional knitted fabric (Trade Name: FUSION; Model Number: AKE69440; Distributor: ASAHI KASEI FIBERS CORPORATION; Thickness: 4.3 mm; Unit Weight: 321 g/m²; made from polyamide) which has knitted structure shown in FIG. 3 was cut into a circular form having a diameter of 5 cm. Next, an acetone solution of 120 mg of 2,3,5,6-tetrafluorobenzyl(1R)-trans-3-(2,2-dichlorovinyl)-2,2-dimethylcyclopropanecarboxylate was applied uniformly to the three-dimensional knitted fabric. And then, acetone was air-dried to prepare support member 102 of the present invention.

### Comparison Example 1

First, a carrier which was used in an insect-pest-control apparatus of a non-heating type (Trade Name: Osotode-No-Mat; produced by Earth Chemical Co., Ltd.; commercially available) was cut into a circular form having a diameter of 5 cm. The carrier was constructed with three overlapped nets. Each net was made of twisted yarns. The carrier had a thickness of 0.7 mm and a unit weight of 210 g/m². The carrier was made from polyester. Next, an acetone solution of 120 mg of 2,3,5,6-tetrafluoro-4-methoxymethylbenzyl(1R)-trans-3-(1-propenyl(Z/E=8/1))-2,2-dimethylcyclopropanecarboxylate was applied uniformly to the carrier. And then, acetone was air-dried to prepare support member 101 for comparison.

### Comparison Example 2

First, a carrier which was used in an insect-pest-control apparatus of a non-heating type (Trade Name: Osotode-No-Mat; produced by Earth Chemical Co., Ltd.; commercially available) was cut into a circular form having a diameter of 5 cm. The carrier was constructed with three overlapped nets. Each net was made of twisted yarns. The carrier had a thickness of 0.7 mm and a unit weight of 210 g/m². The carrier was made from polyester. Next, an acetone solution of 120 mg of 2,3,5,6-tetrafluorobenzyl(1R)-trans-3-(2,2-dichlorovinyl)-2,2-dimethylcyclopropanecarboxylate was applied uniformly to the carrier. And then, acetone was air-dried to prepare support member 102 for comparison.

### Test Example 1

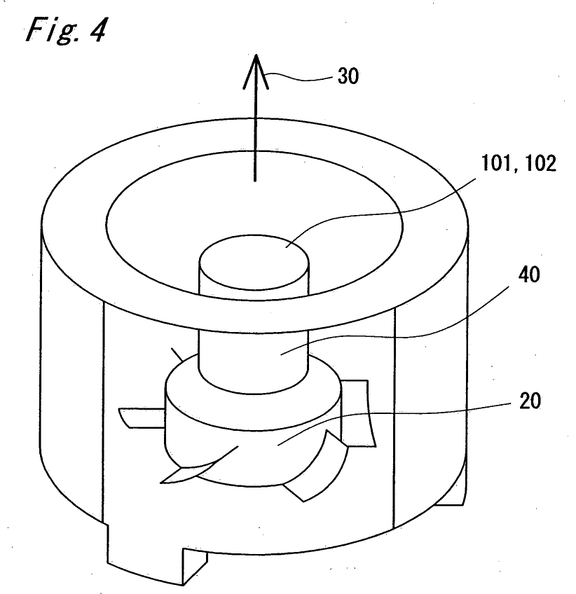

The support member 101 of Example 1 was used to prepare a test apparatus shown in FIG. 4. In the test apparatus, electric fan 20 was placed below plastic cylinder 40. The support member 101 was placed on an upper part of the cylinder 40 so that airflow 30 from the fan 20 might hit perpendicularly to a planar surface of the support member 101. The cylinder 40 had a height of 7 cm and a diameter of 8.3 cm.

The support member 101 of Comparison Example 1 was used in the same manner to prepare the test apparatus.

Insecticidal tests were performed as follows. First, five female adult insects of Culex pipiens pallens were released into the glass tube and both ends of the glass tube were closed by nylon nets. The glass tube had a diameter of 4 cm and a height of 12 cm. Second, the glass tube was set in the plastic cylinder and then a metal cylinder was placed under the plastic cylinder. The plastic cylinder had a diameter of 18 cm and a height of 30 cm. The metal cylinder had a diameter of 20 cm and a height of 80 cm. Third, the above-mentioned test apparatus was placed at a bottom of the metal cylinder and then the electric fan was activated so that a speed of airflow passing through the support member might be 1.0 m/s. Finally, after 1 minute from activating the electric fan, a number of knock-downed insects of Culex pipiens pallens was counted. And then, a knock-downed rate was calculated. This result is shown in Table 1.

TABLE 1

| | knock-downed rate (%) |
|---|---|
| Support member | after 1 minute |
| Example 1 | 100 |
| Comparison Example 1 | 20 |

### Test Example 2

The support member 102 of Example 2 was used to prepare the test apparatus shown in FIG. 4. In this test apparatus, the electric fan 20 was placed below the plastic cylinder 40. The support member 102 was placed on the upper part of the cylinder 40 so that airflow 30 from the fan 20 might hit perpendicularly to a planar surface of the support member 102. The cylinder 40 had a height of 7 cm and a diameter of 8.3 cm.

The support member 102 of Comparison Example 2 was used in the same manner to prepare the test apparatus.

Insecticidal tests were performed as follows. First, five female adult insects of Culex pipiens pallens were released into a glass tube and both ends of the glass tube were closed by nylon nets. The glass tube had a diameter of 4 cm and a height of 12 cm. Second, the glass tube was set in a plastic cylinder and then a metal cylinder was placed under the plastic cylinder. The plastic cylinder had a diameter of 18 cm and a height of 30 cm. The metal cylinder had a diameter of 20 cm and a height of 40 cm. Third, the above-mentioned test apparatus was placed at a bottom of the metal cylinder and then the electric fan was activated so that a speed of airflow passing through the support member might be 1.0 m/s. Finally, after 1 minute and 5 minutes from activating the electric fan, a number of knock-downed insects of Culex pipiens pallens was counted. And then, a knock-downed rate was calculated. This result is shown in Table 2.

TABLE 2

| | knock-downed rate (%) |
|---|---|
| Support member | after 1 minute |
| Example 2 | 100 |
| Comparison Example 2 | 20 |

1-5. (canceled)

6. A method for controlling insects by using blowing air, comprising blowing air through a support member that comprises a carrier that includes a three-dimensional knitted fabric having a first knitted fabric layer, a second knitted fabric layer, and connecting yarns interconnecting the first knitted fabric layer and the second knitted fabric layer, with a plurality of the connecting yarns extending from each stitch of the first knitted fabric layer to a corresponding stitch of the second knitted fabric layer, and with at least one of the first and second knitted fabric layers having a mesh-like structure, and a volatile insect-pest-control component that is impregnated in the carrier or adhered to the carrier.

7. The method of claim 6, wherein each stitch of the first knitted fabric layer and the corresponding stitch of the second knitted fabric layer do not face each other.

8. The method of claim 7, wherein the first knitted fabric layer, the second knitted fabric layer, and the connecting yarns comprise polyamide fibers.

9. The method of claim 6, wherein the first knitted fabric layer, the second knitted fabric layer, and the connecting yarns comprise polyamide fibers.

10. The method of claim 6, wherein said blowing comprises blowing air through the support member using a fan.

11. The method of claim 10, wherein said blowing comprises the fan blowing air through the support member at a speed of 0.1 to 10 m/s.
